(19) United States
(12) Patent Application Publication
(10) Pub. No.: US 2011/0238020 A1
(43) Pub. Date: Sep. 29, 2011

(54) OPTICAL MEASUREMENT CATHETER FOR THERMODILUTION MEASUREMENT AND PULSE CONTOUR ANALYSIS

(75) Inventors: Oliver Goedje, Strasslach (DE); Marcus Veeck, Koblenz (DE); Robert Herz, Haar (DE); Matthias Bohn, Munich (DE); Stephan Joeken, Schopfheim (DE)

(73) Assignee: PULSION Medical systems AG, Munich (DE)

(21) Appl. No.: 12/737,953

(22) PCT Filed: Sep. 2, 2009
(86) PCT No.: PCT/EP2009/061314

§ 371 Date: May 27, 2011

(30) Foreign Application Priority Data
Sep. 4, 2008 (DE) 10 2008 045 719.1
Nov. 5, 2008 (DE) 10 2008 055 952.0

(51) Int. Cl. A61M 5/32; A61B 5/00

(52) U.S. Cl. 604/272; 604/264; 600/309

(57) ABSTRACT

The invention relates to a catheter device with an integrated fiber optic, in particular for use in thermodilution measurement and pulse contour analysis. The device comprises an arterial catheter (2) with a suitable intravascular part and a suitable extravascular part and an optical sensor unit for combined pressure and temperature measurement at a measurement location at the distal end of the suitable intravascular part or proximate to the distal end of the proper intravascular part of the catheter (2). The optical sensor unit comprises a fiber optic conductor (11) that runs from the measurement location to a proximal port.

The invention relates to a catheter device for measurement of pressure and temperature. In particular, the invention relates to a catheter device for use in thermodilution measurements and pulse contour analysis.

Catheter devices for measurement of pressure and temperature are known from the prior art, in various embodiments.

Devices for determining hemodynamic parameters from a dilution curve obtained by means of invasive measurements are widespread, particularly in intensive-care medicine. In this connection, the hemodynamic parameters are, above all, characteristic volumes or volume flows such as cardiac output (Cardiac Output, CO), global end-diastolic volume (GEDV), and the volume of the extravascular lung water (EVLW). Corresponding systems are commercially available and mostly work with cold (i.e. a cooled bolus) as an indicator. Therein, a defined amount of cold fluid is injected into the test subject and the temperature progression of the blood at a different location of the circulation is recorded by way of a temperature probe. Aside from the widespread right-heart catheter systems, with which thermodilution measurements are carried out in the pulmonary artery as the measurement location, systems for transpulmonary thermodilution measurements have established themselves on the market.

For measurement of temperature in thermodilution methods, a thermistor, i.e. a resistance temperature sensor (RTD, Resistance Temperature Detector) is used. RTDs are popular because of their stability and their great accuracy, and provide an essentially linear measurement signal.

Methods and devices for transpulmonary thermodilution measurements are disclosed in, inter alii, U.S. Pat. No. 5,526,817 A and U.S. Pat. No. 6,394,961 B1, as well as the literature cited therein.

U.S. Pat. No. 5,526,817 A describes a method for determining the circulatory filling state by means of thermodilution. Therein, in order to assess the circulatory filling state of a patient, particularly of the global end-diastolic volume (GEDV), of the intrathoracic blood volume (ITBV), of the pulmonary blood volume (PBV), of the extravascular lung water volume (EVLW) and/or of the global cardiac function index (CFI), the intrathoracic thermovolume (ITTV) and the pulmonary thermovolume (PTV) are determined.

Pulse contour analysis is a method for semi-invasive determination, in particular of the cardiac output. Therein, the pulse volume of the heart is calculated from the shape of an arterial blood pressure curve by means of mathematical methods. This method is based on the extraction and the clinically usable representation of the data contained in the arterial blood pressure curve. The great dependence on the quality of the pressure signal obtained is problematic.

The determination of hemodynamic parameters, particularly of the cardiac output (CO), by means of pulse contour analysis on the basis of a non-linear Windkessel model has been described in detail in DE 198 14 371 A1 as well as in the further literature listed there. The basic measurement variable for pulse contour analysis is a pressure that approximately corresponds to the aortic pressure, which pressure is continuously measured, for example, by means of an arterial catheter in a femoral artery.

Essential variables in the determination of hemodynamic parameters on the basis of the function P(t), i.e. the time progression of the pressure signal that approximately corresponds to the aortic pressure, are, in particular, the systemic vascular resistance (Systemic Vascular Resistance, SVR), as well as, furthermore, the so-called compliance (C). The former is understood to be, in illustrative manner, the flow-through resistance of the vascular system of the systemic circulation, the latter as resilience in the region of the aorta. In a flow schematic, these variables can be represented as resistance and capacitance. In older approaches, in particular, the compliance is sometimes disregarded.

The determination of the blood pressure required for pulse contour analysis generally is performed using well known membrane pressure transducers.

The pressure sensors known from the state of the art are disposed on the proximal end of the respective catheter or of a pressure tube attached thereto. In this connection, the fluid column in the catheter lumen transmits the arterial pressure to the membrane of the pressure sensor.

In order to obtain precise measurement values of the blood pressure, the catheter lumen must have a minimum diameter in the catheter. As the lumen diameter decreases, pressure losses occur as a result of the friction forces that occur at the lumen wall, and as a result of the inflow pressure loss. In order to obtain the most precise possible measurement values from membrane pressure measurements, with little pressure loss, the catheter requires a correspondingly dimensioned catheter lumen. The friction of the blood fluid in the catheter lumen also brings about an attenuation, i.e. a reduction in the measured amplitude of the pressure variations that occur. The attenuation distorts the pressure measurement, particularly at low pressure variations, and brings about a reduced signal/noise ratio. Further attenuation effects and effects that impair the measurement can occur as a result of air inclusions or outgassing.

In a pressure sensor measurement, the hydrostatic pressure between the proximally disposed pressure membrane and the height of the distal catheter end is also measured, and must therefore be corrected out. This requires a zero adjustment. An impairment of the measurement values occurs when the body part on which the catheter device is fixed in place and the pressure sensor unit are moved relative to one another in terms of their height. A remedy can be provided by determining the height difference between the relative position of the patient and the measurement unit, or by repeating the zero adjustment.

Thermodilution and pulse contour methods are often combined. For one thing, in this way a thermodilution measurement can be used for calibrating the pulse contour method, for another, many parameters, such as the extravascular lung water, for example, are not accessible to measurement by means of a pulse contour analysis method. Pulse contour analysis offers the advantage of a continuous measurement, whereas cold bolus injection, of course, cannot be administered continuously. A device for combined use of thermodilution method and pulse contour method is commercially available, among others, from PULSION Medical Systems AG, under the name PiCCO.

According to the state of the art, it is generally possible to carry out the pressure and temperature measurements required for thermodilution and pulse contour analysis methods using catheters in larger blood vessels. As a rule, these are combined pressure/temperature measurement catheters having an integrated thermistor and a pressure lumen for connection to a pressure sensor. For smaller blood vessels such as the arteria radialis, this applies only with restrictions: While a signal that can be used for pulse contour analysis can certainly be obtained from the arteria radialis, it is hardly possible to carry out thermodilution measurements in the radial artery or other small arteries by means of conventional catheters, over an extended period of time. After a conventional catheter has been introduced into the radial artery, long-lasting blood vessel contractions frequently occur, and thus the blood flow in the artery is (almost) stopped. Interruption of the blood flow makes thermodilution measurements impossible. Only by means of actively suctioning away the blood measurement values can be determined, under some circumstances, but it must be assumed that distortion occurs then. Pressure and temperature measurements in the arteria radialis would have the advantage that the arteria radialis of the patient is generally more easily accessible to the medical personnel than in the case of a puncture of the arteria femoralis, for example.

The underlying object of the present invention is therefore providing a device for measurement of pressure and temperature within the body of a living being, so that the requirement of a zero adjustment is avoided as far as possible for determining pressure, that a possible blood stasis is prevented even when used in smaller arteries, and that the signal quality for the pulse contour analysis is improved.

According to one aspect of the invention, this abject is achieved with the device according to claim 1. Further embodiments are recited in the dependent claims.

The invention accordingly relates to a catheter device for measurement of pressure and/or temperature, preferably a combined pressure and temperature measurement. Therein, the device has an arterial catheter having an, according to its intended purpose, intravascular part, and an, according to its intended purpose, extravascular part, as well as an optical sensor unit for measurement, preferably combined measurement, of pressure and/or temperature, at a measurement location, at the distal end of the, according to its intended purpose, intravascular part or in the vicinity of the distal end of the, according to its intended purpose, intravascular part. In this connection, the optical sensor unit has a fiber-optic duct, which runs from the measurement location to a proximal port. In place of the preferred combined pressure/temperature measurement unit, according to another advantageous embodiment, separate pressure and temperature sensor units can also be provided, preferably both optical, but alternatively, also one of them conventional. In particular, the combination of an optical pressure measurement unit and temperature measurement by means of a thermistor can be advantageous, because here, the attenuation effects mentioned above are reliably avoided in the pressure measurement, but the experience from conventional measurements (calibration data, use of proven measurement transformers, etc.) can still be used for the temperature measurement.

The combined pressure and temperature measurement according to the principle of Fabry-Perot interferometry is particularly suitable for implementation of the invention. Therein, the interferences resulting from the deflection of a reflective membrane that is affixed in front of the partially permeable mirrored fiber end are evaluated (for example by means of spectrometry). The temperature and/or pressure measurement can also take place using a luminescent element that is dependent on temperature or pressure. The sensor can also be configured as an optical sensor like the one known from U.S. Pat. No. 4,986,671. The pressure is measured at the tip of the sensor fiber, by means of reflected light from a convex surface whose curvature changes in response to a change in the pressure. Therein, the amount of reflected light correlates with the pressure against the surface at the sensor. Furthermore, the sensor unit can also be formed by a self measuring elastic fiber for detecting temperature and pressure. Self measuring elastic fibers are known, for example, from WO 2007/003876.

Temperature and pressure measurements can generally be determined by means of a combined optical element. However, a two-fiber configuration is also possible, for example, in which the temperature is measured at a distal end of one sensor fiber, and the pressure is measured at the distal end of another sensor fiber. Thus, the above measurement methods, by means of membrane deflection determined using interferometry, membrane curvature, fiber deformation, and luminescent elements, can also be combined with one another.

The thermistors for temperature measurement known from the prior art are thereby replaced, according to the invention, by the optical sensor, which performs not only the temperature measurement but also the task of measuring the pressure in the punctured artery. A further (pressure measurement) catheter lumen is thus dispensable, or does no longer have to have the diameter that would be required for a conventional pressure measurement to be low in attenuation and pressure loss. The catheter cross-section can therefore be made smaller.

In contrast to the blood pressure measurement by means of an external pressure sensor measurement unit, the measurement values are measured, according to the invention, by means of the thermodilution catheter device, in situ, directly at the respective measurement location, in other words in the blood vessel. A zero adjustment may therefore be dispensable.

Preferably, the arterial catheter is configured as a radialis catheter.

Preferably, the catheter device has an outside diameter, on the, according to its intended purpose, intravascular part of the arterial catheter, of at most 1.67 mm, particularly preferably at most 1.5 mm. The introduction of large-lumen catheters into the arteria radialis for measurement of pressure or temperature is thereby avoided. Now, instead, small-lumen catheters with integrated pressure and temperature sensors are provided, which are much thinner than conventional measurement catheters. The accompanying impairment of the blood flow in the the artery after the puncture has taken place is thus clearly less than in the case of conventional catheters.

According to an advantageous enhancement, the catheter device furthermore has an introduction aid through which the arterial catheter can be pushed. In this connection, the length of the introduction aid is coordinated with the length of the catheter, in such a manner that the introduction aid can be positioned completely proximal to the, according to its intended purpose, intravascular part of the catheter, by displacing the introduction aid and the catheter relative to one another. The introduction aid can therefore be pulled out as soon as the catheter is placed as intended.

Preferably, the length of the introduction aid of the catheter device amounts to at most half the length of the arterial catheter.

Particularly preferably, the introduction aid of the catheter device is configured as a puncture cannula.

After puncture at the radial artery has taken place, the small-lumen catheter is introduced through the hollow structure of the introduction aid, up to the desired length. After positioning of the catheter in the artery, the introduction aid is removed from the patient, so that there is no unnecessary stress on the puncture site.

According to an advantageous enhancement, the introduction aid and the arterial catheter of the catheter device are packed together in sterile packaging. This has the advantage that the combined introduction aid and arterial catheter device can be hygienically applied to the patient by the medical personnel, with the least possible expenditure of time. “Threading” the catheter in can be eliminated.

According to an advantageous embodiment, the sensor unit is integrated into the arterial catheter. After introduction of the catheter into an artery, the sensor unit is immediately ready for measurement of pressure or temperature, without unnecessary introduction into a catheter lumen. The risk of introduction of germs is also reduced, if no separate sensor is introduced into a lumen.

According to an advantageous enhancement, the arterial catheter of the catheter device has a lumen that connects an opening disposed at the distal end of the, according to its intended purpose, intravascular part or in the vicinity of the distal end of the, according to its intended purpose, intravascular part with a proximal port. Such a lumen can be used for taking blood samples, for example, or for injection of required substances.

According to an alternative embodiment, the sensor unit of the catheter device is configured as a probe that can be pushed into a probe lumen of the arterial catheter. Thus, a measurement probe can be introduced into a catheter that is perhaps already in place for other reasons.

For example, a connector having a Y-shaped construction is used for introduction of the lumen probe into the catheter device. The lumen probe is introduced through a branch at the port. After the catheter has been put into place according to the Seldinger technique, the probe should be introduced into the probe lumen of the catheter. However, the device according to the invention is not generally restricted to introduction by means of Seldinger wires.

According to an advantageous enhancement, the probe can be fixed in place on the arterial catheter in a position that is defined relative to the probe lumen. By fixing the probe accordingly, it is avoided that the latter is accidentally pulled away from the measurement site. Furthermore, it is avoided that contaminating germs are introduced by pushing the probe farther in. For introducing the probe into the catheter lumen, first the required probe length or introduction length is determined. The catheter device has a port that allows advancing the measurement probe into the catheter lumen. The suitable length is to be set using, for example, markings on the probe. Subsequently, the optical probe is fixed in place at the port: It is also possible to use a probe having attachment means firmly connected with the probe, which means result in a predetermined position of the distal probe end at a given catheter length. In this case, probe and catheter need to be coordinated with one another.

Preferably, single-use closure means are provided for fixing the probe in place relative to the arterial catheter, which means cannot be released without destroying them, after the probe has been fixed in place. This adds to making accidental advancement or removal of the sensor more difficult.

According to an advantageous enhancement, the arterial catheter is configured to have one lumen.

According to an advantageous enhancement, the cross-sectional area of the sensor lumen amounts to at least twice the cross-sectional area of the probe in the catheter device. In this way, blood samples can be removed from the lumen, for example, while the probe is in place, if necessary.

According to an advantageous enhancement, the arterial catheter comprises a lumen that is separate from the probe lumen, which lumen connects a further proximal port with an opening disposed at the distal end of the, according to its intended purpose, intravascular part or in the vicinity of the distal end of the, according to its intended purpose, intravascular part. Such a lumen can be used for taking blood samples, for example, or for injection of required substances.

In addition to the introduced optical probe, a pressure tube for taking blood can be connected. A conventional pressure sensor can also be connected, for example for calibration or comparison measurement.

Alternatively, in addition to the Y-shaped coupling piece mentioned above, coupling pieces configured in a different manner can also be provided on the catheter device.

According to another aspect of the invention, a device for determining hemodynamic parameters has a catheter device of the above type and an evaluation unit that can be connected with the sensor unit and is set up to calculate hemodynamic parameters using measurement signals obtained by means of the sensor unit. In this connection, the calculation can take place using known thermodilution measurement algorithms and known pulse contour analysis algorithms.

According to an advantageous enhancement, the device furthermore has a central venous catheter having means for introducing a temperature change into central venous blood, whereby the evaluation unit is adapted to implement a calculation algorithm for transpulmonary thermodilution methods in the calculation of at least one of the hemodynamic parameters.

Preferably, the means for introducing a temperature change into central venous blood comprise an injection channel.

According to an advantageous enhancement, the injection channel of the device has means for obtaining a measurement signal for determining an injection time, which means can be connected with the evaluation unit. Therein, the evaluation unit is adapted to determine the injection time from the measurement signal. Corresponding injection channels and methods are known from, inter alii, U.S. Pat. No. 6,491,640 and U.S. Pat. No. 6,200,301.

According to an advantageous enhancement, the evaluation unit is set up to implement a pulse contour algorithm in the calculation of at least one of the hemodynamic parameters.

In the following, the invention will be described in greater detail and in the manner of examples, with reference to the drawings. Several preferred embodiments are described, however, the invention is not limited thereto.

Generally, any variant of the invention described or suggested in the present application can be particularly advantageous, depending on the economic and technical conditions of an individual case. To the extent that nothing is said to the contrary, or to the extent that this can basically be implemented technically, individual features of the embodiments described can be interchanged or combined with one another, as well as with features known per se from the prior art.

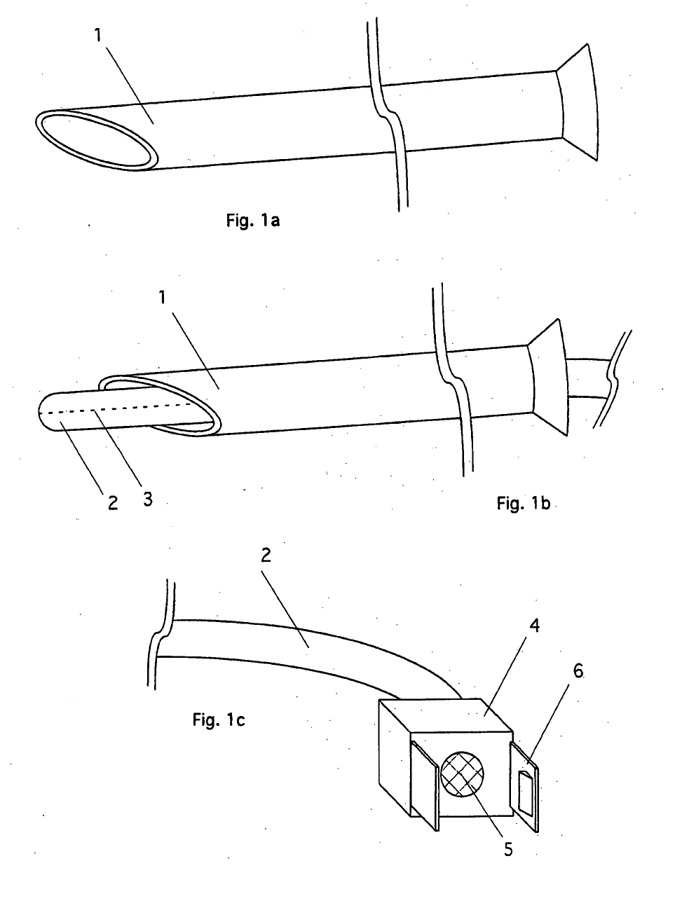

FIG. 1a schematically shows an introduction aid of a catheter device according to the invention,

FIG. 1b schematically shows an arterial catheter according to the invention, with integrated sensor optics, in an introduction aid,

FIG. 1c schematically shows an optical plug of the device according to the invention,

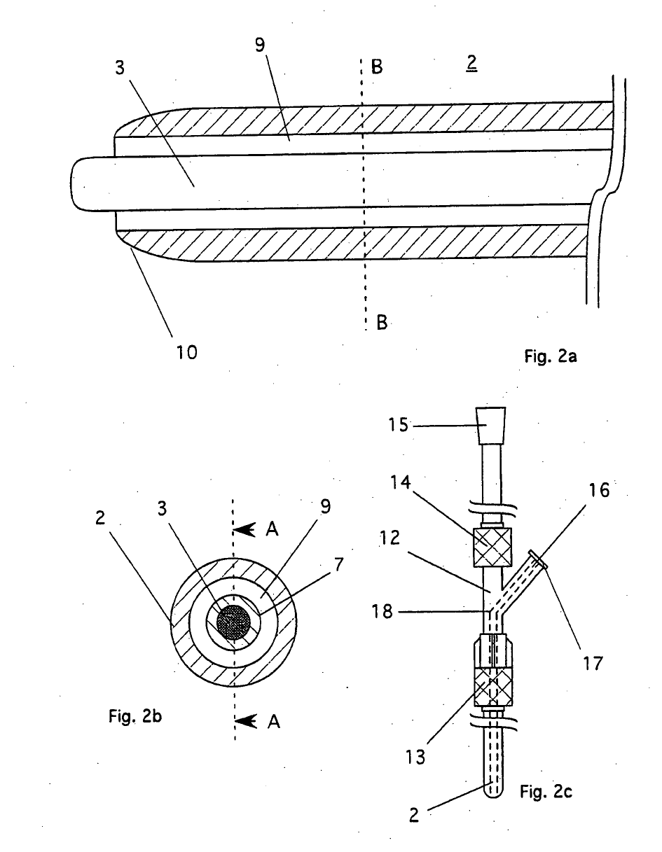

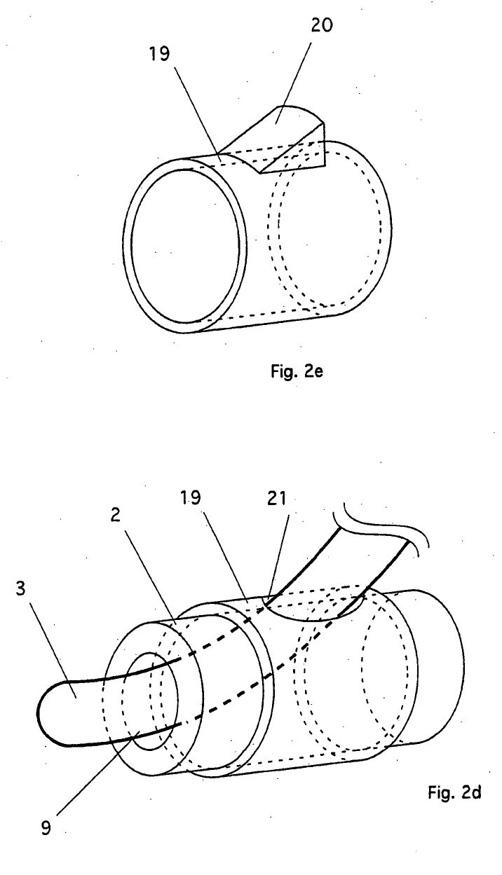

FIG. 2a schematically shows a longitudinal sectional view A-A (depicted broken off) of the arterial catheter according to the invention of FIG. 2b, with a probe introduced into a probe lumen,

FIG. 2b schematically shows a cross-sectional view B-B of the arterial catheter according to the invention from FIG. 2a, with the probe introduced,

FIG. 2c schematically shows a coupling piece at the proximal port of the arterial catheter according to the invention,

FIG. 2d schematically shows the alternative coupling piece of the arterial catheter according to the invention, in a spatial view, which piece is affixed to an arterial catheter,

FIG. 2e schematically shows, in a spatial view, a part of an alternative coupling piece of the arterial catheter according to the invention, which coupling piece is structured in a manner similar to FIG. 2d,

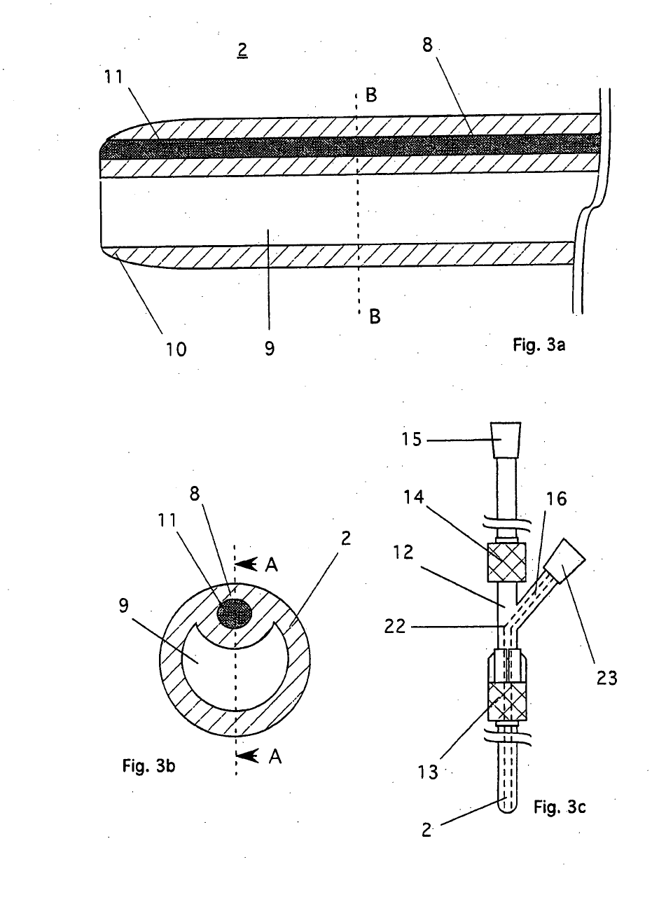

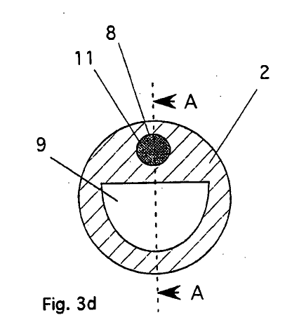

FIG. 3a schematically shows a longitudinal sectional view A-A of the arterial catheter according to the invention, from FIG. 3b, with integrated fiber optics,

FIG. 3b schematically shows a cross-sectional view B-B of the arterial catheter according to the invention, from FIG. 3a, with integrated fiber optics, and

FIG. 3d schematically shows an alternative cross-sectional view of an arterial catheter according to the invention, similar to FIG. 3a.

FIG. 1a shows an introduction aid of a catheter device according to the invention. The introduction aid is a hollow, short cannula 1. An artery, for example the arteria radialis, is punctured on the patient using the cannula 1.

After the puncture, the thermodilution catheter 2 is introduced into the cannula 1. FIG. 1b schematically shows a catheter device according to the invention for obtaining measurement data for pulse contour analysis and thermodilution measurement. The catheter 2 can be positioned at the desired arterial measurement site carefully advancing the catheter 2 into the hollow, short introduction aid. In order to keep the injury due to puncture as little as possible, the short cannula 1 is removed from the artery and from the lower arm of the patient after advancing of the catheter 2 has taken place, and the catheter 2 is fixed in place at the measurement site. The free flow of blood in the artery is not markedly influenced despite the catheter 2 with the integrated optics 3 that has been introduced into the artery. Subsequent to removal of the introduction aid, the actual measurement of the pressure and temperature progressions in the bloodstream can take place using the integrated sensor unit. Therein, the actual sensor element, for example configured according to the principle of a Fabry-Perot interferometer, is disposed at the distal tip of the integrated fiber optics 3.

FIG. 1c schematically shows a plug 4 of the catheter device according to the invention. An optical contact surface 5 is situated at the face side of the optical plug 4, by way of which surface incident and outgoing light is passed on to a second coupling plug (not shown). To connect the coupling plugs with one another, the plug 4 has a clip-on mechanism 6, for example, for accommodating the second coupling plug. The second coupling plug is configured as a counterpart to the coupling piece 4, and connected with a light source and a detector (both not shown), the latter of which converts the system response of the sensor element to the light radiated in into electrical signals that can be evaluated. For this purpose, depending on the measurement principle applied (see above), light sources and detectors known from the state of the art can generally be used.

An evaluation unit processes the electrical signals generated by the detector and calculates the pressure and temperature values from these, and the desired physiological parameters from their progression. Therein, the pressure and temperature progressions determined by way of optical measurement methods can be evaluated by means of known thermodilution and pulse contour algorithms. Light source and detector can be combined into an optical module that is integrated into a patient monitor or can be connected to a patient monitor. When a suitable measurement transformer is used, the optical module can be configured in such a manner that its output signals correspond to the output signals of conventional pressure and temperature sensors. Thus, the catheter device according to the invention can also be configured as a retrofitting solution for conventional patient monitors.

FIG. 2a schematically shows a longitudinal section A-A (broken off) of the arterial catheter 2 from FIG. 2b, with an introduced probe 3. During introduction, the probe 3 is guided through the catheter lumen 9 of the catheter 2. Therein, the advanced probe 3 is disposed on the catheter 2 in such a manner that it runs parallel in the catheter lumen 9, in the longitudinal direction. The measurement probe 3 exits from the catheter lumen 9 at the distal end 10 of the catheter 2.

FIG. 2b schematically shows a cross-section B-B of the arterial catheter 2 from FIG. 2a, with a probe 3 introduced. The cross-sectional area of the catheter lumen 9 corresponds, here, to slightly more than twice the cross-section of the advanced probe 3. The probe 3 furthermore has a mantling 7. The sensor optics or the sensor measurement technology corresponds to the catheter measurement technology described above.

FIG. 2c schematically shows a port of the arterial catheter 2 for introduction of the probe 3. The port is connected with a Y-shaped coupling piece 12. The coupling piece 12 has a connection location 13 and 14, respectively, at its distal and proximal end, in the longitudinal direction. The proximal end of the catheter 2 introduced into the artery is connected at the distal connection location 13 of the coupling piece 12. Connecting a pressure tube 15, an injection syringe, a flushing device, or the like, is enabled at the proximal connection location 14 of the coupling piece 12. The pressure tube 15 is intended for taking blood or for a conventional pressure measurement. The connection locations 13 and 14 can be configured, for example, as Luer lock adapters, respectively. The branch 16 of the coupling piece 12 that protrudes in the longitudinal direction has a shaft 18 for introduction of the lumen probe 3 into the catheter lumen 9. The guide shaft 18 of the probe 3 to be introduced is indicated by means of broken lines. A flange 17 forms the end of the protruding branch 16, on which, in the actual technical implementation, suitable connection means for a locally fixed connection of the probe 3 to the coupling piece 12 are provided, in advantageous manner.

For example, the probe 3 can be glued into a connection piece that can be connected with the coupling piece by way of a Luer lock connection. Furthermore, a clamp connection can be provided, but this should be advantageously implemented in such a manner that, as far as possible, no clamping forces occur that could damage the probe 3. This can be implemented by means of elastic clamping bodies having a sufficiently large area, which bodies can be configured, for example, as an inner cone/outer cone pairing of parts that can be moved longitudinally into one another. A restriction in clamping force can be implemented by means of elements that shear off or (approximately in the manner of a torque wrench) slip through.

Aside from the arrangement shown, an arrangement is also possible in which the probe 3 is introduced from the direction of the connection location 14, and the branch 16 serves as a connector for flushing or the like. Embodiments in which the probe is firmly connected with the coupling piece 12 right from the start, for example glued into it, are also possible.

FIG. 2d schematically shows an alternative coupling piece 19 to the coupling piece 12 from FIG. 2c, in a spatial view; this piece is structured in particularly simple manner. The coupling piece 19 is configured in the shape of a ring-shaped cuff that can be rotated in such a manner that the opening 21 in the cuff 19 covers an opening in the catheter 2, so that the probe 3 can be pushed into the catheter lumen 9. For protection against contamination, the opening 21 can be protected with a septum, for example.

The wedge-shaped attachment 20 in FIG. 2e is supposed to schematically indicate that the opening 21 does not, of course, have to be configured as a simple hole, possibly closed off by a septum, but rather can be provided with an introduction aid 20 for the probe 3, as indicated in schematical manner, furthermore with attachment means for fixation of the sensor, closures, or more of the like.

The longitudinal section shown schematically in FIG. 3a (along the section line A-A in FIG. 3b) of the device of an arterial catheter 2, according to the invention, has a separate optics lumen 8 into which the fiber-optic conductor 11 is firmly integrated. A further catheter lumen 9 runs parallel to the optics lumen 8. At the distal end 10 of the catheter 2, the sensor optics 11 essentially end flush with the optics lumen 8.

FIG. 3b schematically shows a cross-section (along the section line B-B in FIG. 3a) of the arterial catheter 2 from FIG. 3a.

FIG. 3c schematically shows a coupling piece 12 of an arterial catheter 2 with integrated fiber optics 11, configured as in FIGS. 3a and 3b, for example. The structure is similar to that in FIG. 2c, as a Y-shaped coupling piece 12. The coupling piece 12 has a connection location 13 and 14 at its distal and proximal end, respectively, in the longitudinal direction. At the distal coupling location 13 of the coupling piece 12, the latter is firmly connected with the proximal end of the catheter 2. At the proximal connection location 14 of the coupling piece 12, it is possible to connect a pressure tube 15 or the like. The connection location 14 is configured as a Luer lock adapter, for example. The optics 11 are integrated into the shaft 22 of the branch 16 of the coupling piece 12, which branch protrudes in the longitudinal direction. The shaft 22 of the fiber-optic conductor 11 is shown in the form of a broken line. A plug 23 forms the end of the protruding branch 16, in order to be able to connect light source and detector for the optical response signal of the sensor here.

FIG. 3d shows a variant of the arterial catheter 2 from FIG. 3a with integrated fiber optics 11. In FIG. 3d, a catheter lumen 9 in the form of a semicircle is provided next to the optics lumen 8 of the catheter 2.

1. A catheter device, having an arterial catheter having an intravascular part and an extravascular part as well as an optical sensor unit for measurement of pressure and/or temperature, at a measurement location, at the distal end of the intravascular part or in the vicinity of the distal end of the intravascular part wherein the optical sensor unit comprises a fiber-optic duct, which runs from the measurement location to a proximal port.

2. Catheter device according to claim 1, wherein the optical sensor unit is a sensor unit for combined measurement of pressure and temperature.

3. Catheter device according to claim 1, wherein the arterial catheter is configured as a radialis catheter.

4. Catheter device according to claim 1, wherein the intravascular part of the arterial catheter, has an outside diameter of at most 1.67 mm.

5. Catheter device according to claim 4, wherein the intravascular part of the arterial catheter, has an outside diameter of at most 1.67 mm.

6. Catheter device according to claim 1, which furthermore has an introduction aid through which the arterial catheter can be pushed, wherein the length of the catheter and of the introduction aid are coordinated with one another in such a manner that positioning in which the introduction aid is disposed completely proximal to the intravascular part of the catheter, can be achieved, by displacing the introduction aid and the catheter relative to one another.

7. Catheter device according to claim 6, wherein the length of the introduction aid amounts to at most half the length of the arterial catheter.

8. Catheter device according to claim 6, wherein the introduction aid is implemented as a puncture cannula.

9. Catheter device according to claim 6, wherein the introduction aid and the arterial catheter are packaged together, in sterile packaging.

10. Catheter device according to claim 1, wherein the sensor unit is integrated into the arterial catheter.

11. Catheter device according to claim 10, wherein the arterial catheter has a lumen that connects an opening disposed at the distal end of the intravascular part or in the vicinity of the distal end of the intravascular part with a further proximal port.

12. Catheter device according to claim 1, wherein the sensor unit is structured as a probe that can be pushed into a probe lumen of the arterial catheter.

13. Catheter device according to claim 12, wherein the probe can be fixed in place on the arterial catheter in a defined position relative to the sensor lumen.

14. Catheter device according to claim 13, wherein single-use closure means are provided for the fixation of the probe relative to the arterial catheter, which means cannot be loosened, after fixation of the probe, without destroying them.

15. Catheter device according to claim 12, wherein the arterial catheter is structured to have one lumen.

16. Catheter device according to claim 15, wherein the cross-sectional area of the probe lumen amounts to at least twice the cross-sectional area of the sensor.

17. Catheter device according to claim 12, wherein the arterial catheter has a lumen that is separate from the probe lumen and connects an opening disposed at the distal end of the intravascular part or in the vicinity of the distal end of the intravascular part with a further proximal port.

18. Device for determining hemodynamic parameters, comprising a catheter device according to claim 1, and an evaluation unit that can be connected with the sensor unit, which evaluation unit is adapted to calculate hemodynamic parameters, using measurement signals obtained by the sensor unit.

19. Device according to claim 18, furthermore having a central venous catheter having means for introducing a temperature change into central venous blood, wherein the evaluation unit is adapted to implement a calculation algorithm for transpulmonary thermodilution methods in the calculation of at least one of the hemodynamic parameters.

20. Device according to claim 19, wherein the means for introducing a temperature change into central venous blood comprise an injection channel.

21. Device according to claim 20, wherein the injection channel has means for obtaining a measurement signal to determine an injection time, which means can be connected with the evaluation unit, wherein the evaluation unit is set up to determine the injection time from the measurement signal.

22. Device according to claim 18, wherein the evaluation unit is adapted to implement a pulse contour algorithm in the calculation of at least one of the hemodynamic parameters.
